(19) United States
(12) Patent Application Publication
(10) Pub. No.: US 2022/0226509 A1
(43) Pub. Date: Jul. 21, 2022

(54) HIGH-BRIGHTNESS NANODOT FLUOROPHORES BY COVALENT FUNCTIONALIZATION

(71) Applicant: Michigan Technological University, Houghton, MI (US)

(72) Inventors: Yoke Khin Yap, Houghton, MI (US); Dongyan Zhang, Houghton, MI (US); Amit Acharya, Houghton, MI (US); Nazmiye Yapici, South Lyon, MI (US); Xiuling Liu, Ann Arbor, MI (US)

(21) Appl. No.: 17/615,425

(22) PCT Filed: Jun. 1, 2020
(86) PCT No.: PCT/US2020/035568

§ 371 Date: Nov. 30, 2021

Related U.S. Application Data
(63) Continuation of application No. 15/953,200, filed on Apr. 13, 2018.
(60) Provisional application No. 62/485,379, filed on Apr. 13, 2017.
(60) Provisional application No. 62/855,121, filed on May 31, 2019.

(51) Int. Cl. A61K 49/00; C07K 16/28; G01N 33/533; B82Y 5/00; G01N 33/543; G01N 33/50; A61B 5/00

(52) U.S. Cl. CPC A61K 49/0041; A61K 49/0058; C07K 16/2812; A61B 5/0071; B82Y 5/00; G01N 33/54346; G01N 33/5005; G01N 33/533

(57) ABSTRACT

A example compound according to the present disclosure includes, among other possible things, a nanodot carrier, a moiety, and a linker having first and second functional groups, wherein the first functional group is covalently linked to the nanodot carrier, and the second functional group is covalently linked to the moiety. An example method of making a nanodot carrier is also disclosed.

### CROSS-REFERENCE TO RELATED APPLICATIONS

This application claims priority to U.S. Provisional Patent Application Ser. No. 62/855,121 filed May 31, 2019, which is hereby incorporated herein in its entirety. This application is a continuation-in-part of U.S. patent application Ser. No. 15/953,200, filed Apr. 13, 2018, which claims priority to U.S. Provisional Patent Application Ser. No. 62/485,379, filed Apr. 13, 2017, both of which are hereby incorporated herein in their entireties.

### STATEMENT REGARDING FEDERALLY-SPONSORED RESEARCH

The inventions described herein were made with government support under Grant #1261910, Grant #1521057 and Grant #1738466 awarded by the National Science Foundation. The Government has certain rights in this invention.

### BACKGROUND

Fluorophores are compounds with fluorescent properties that have biomedical applications. For example, fluorophores can be used as tracers or dyes for specific staining of certain molecules or structures. More particularly, fluorophores can be used to stain tissues, cells, or materials in a variety of analytical methods, such as fluorescent imaging and spectroscopy.

For the purpose of specific staining, fluorophores should be conjugated with biomolecules such as antibodies. However, reliable tracking and quantification of the fluorophores is challenging due to the low brightness and low photostability of commercial fluorophores. Therefore, a need exists for improved carrier molecules to carry fluorescent entities for biological and other applications. Other biological molecules may also benefit from improved carriers.

### SUMMARY

A example compound according to the present disclosure includes, among other possible things, a nanodot carrier, a moiety, and a linker having first and second functional groups, wherein the first functional group is covalently linked to the nanodot carrier, and the second functional group is covalently linked to the moiety.

An example method of making a nanodot carrier according to the present disclosure includes, among other possible things, mechanically processing nanodots in polar liquid to create imperfections on the nanodots, and treating the nanodots to provide polar groups at the imperfections.

### BRIEF DESCRIPTION OF THE DRAWINGS

FIG. 1A schematically shows an example compound with a nanodot carrier.

FIGS. 1B-1C schematically show synthesis of an example compound like the compound of FIG. 1A from a BN nanodot carrier.

FIG. 2A shows SEM (scanning electron microscopy) images of h-BN bulk powder.

FIG. 2B shows SEM images of h-BN powder after mechanical processing, in this example, treatment with a homogenizer.

FIG. 2C shows TEM (transmission electron microscopy) images of example boron nitride (BN) nanodot carriers.

FIG. 2D shows the excitation-dependent autofluorescence of example BN nanodot carriers and the fluorescence image (inset) under UV lamp.

FIGS. 3A-B show Fourier Transform Infrared Spectroscopy (FITR) results for pristine BN nanodot carriers and processed BN nanodot carriers.

FIG. 4 shows the absorbance spectra of the example fluorophore, pristine carriers, and processed carriers of FIG. 1B.

FIG. 5 shows fluorescence intensity of the example fluorophore, pristine carriers, and processed carriers, and processed carriers with linkers (i.e., functionalized carriers) of FIG. 1B.

### DETAILED DESCRIPTION

Very generally, high-brightness fluorophores contain a carrier element, a fluorescent element, and a linker linking the carrier element to the fluorescent element. For biomedical applications, each of the carrier element, the linker, and the fluorescent element must be biocompatible (though the requirements for biocompatibility will vary with the particular application).

One example carrier element is a processed nanomaterial, such as carbon nanotubes (CNT) and boron nitride nanotubes (BNNTs), both of which can be used for biomedical applications such as cellular drug delivery and spectroscopy applications. However, it has been shown that fluorescent elements linked to carbon nanotubes exhibit quenching, or a reduction in the brightness of the fluorescence.

It has been discovered that certain fluorophores having nanomaterial carriers not only do not exhibit the quenching effect, but also exhibit brightness several orders of magnitude higher than other known fluorophores, as will be discussed herein.

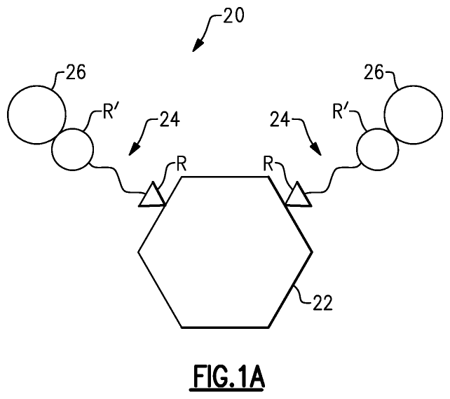

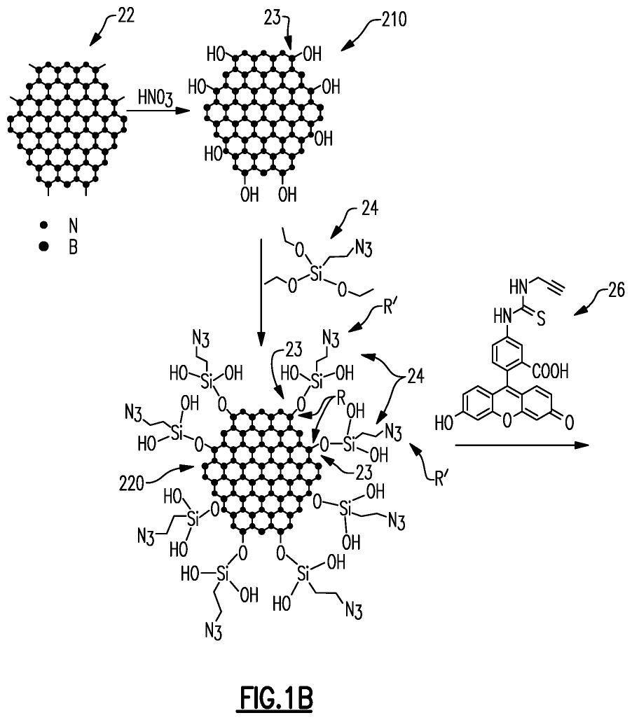

Referring now to FIG. 1A, fluorophore compounds 20 are shown. The compounds 20 generally comprise an inorganic nano-scale (“nanomaterial”) carrier 22, a linker 24, and a moiety 26. In some examples, the compound 20 includes more than one linker 24, and more than one moiety 26.

The carrier 22 is, in one example, a processed BNNT or CNT carrier. In the example of FIG. 1A, the carrier 22 is a zero-dimensional BN “dot” (e.g., the size of the dot in all three dimensions is on the nano-scale, or less than about 100 nm), though carbon dots could also be used. In a more particular example, all three dimensions of a dot carrier are less than about 20 nm. Other example carriers 22 are multi-walled BNNT or CNT carriers, where each BNNT or CNT has multiple co-axial shells of hexagonal boron nitride (h-BN for BNNTs) or graphene (for CNTs), with a typical external diameter of more than about 0.4 nm but less than about 100 nm. The length of these BNNTs and CNTs is between about 1-100 nm. In other examples, the carrier 22 can be another nano-scale inorganic material, such as hexagonal boron nitride (h-BN) nanosheets/nanoparticles, graphene/graphite nanosheets/nanoparticles, molybdenum disulfide (MoS₂) nanosheets/nanoparticles, any transition metal dichalcogenide (TMDCs) nanosheets/nanoparticles, and any nanosheets/nanoparticles of layered materials (materials with covalent layered structures that bond with van der Waals forces between layers).

The linker 24 has two or more functional groups R and R′, as shown in FIGS. 1A-B. The functional groups R and R′ are reactive groups that facilitate covalent bonding of the linker to other structures by any known chemistry. R and R′ can be the same or different functional groups. For example, R and R′ can be ethoxsilane and azide, respectively. R and R′ can be any known functional groups such as amine groups, carboxylic acid, isothiocyanate, maleimide, an alkyne group, a hydroxyl group, a thiol group, monosulfone, or an ester group such as a succinimidyl, sulfodichlorophenol, pentafluorophenyl or tetrafluorophenyl. The linker 24 can be any type of molecule that has two or more functional groups R and R′. One example linker 24 is a linear or branched polymeric molecule. In some examples, the linker 24 has a length of less than about 200 nm. In some examples, multiple linkers 24 can be connected between each other in series.

One functional group R interacts covalently with the carrier 22. A carrier 22 with a linker 24 is known as a “functionalized” carrier 220 as shown in FIG. 1B. That is, when covalently linked to linker 24, the carrier 22/linker 24 structure has a functional group R′ which facilitates covalent bonding of the carrier 22/linker 24 to another moiety 26.

The moiety 26 is, in one example, a fluorescent entity. In this example, the molecule 20 is a fluorophore. The fluorescent entity is any fluorescent dye that is known in the art, including but not limited to coumarins, benzoxadiazoles, acridones, acridines, bisbenzimides, indole, benzoisoquinoline, naphthalene, anthracene, xanthene, pyrene, porphyrin, fluorescein, rhodamine, boron-dipyrromethene (BODIPY) and cyanine derivatives. Many such fluorescent dyes are commercially available. The fluorescent entity can also include tandem dyes which have two different dyes connected and which interact via FRET (fluorescence resonance energy transfer). The fluorescent entity covalently interacts with the functional group R′ of linker 24 as discussed above.

In other examples, moiety 26 is a labelling moiety or other moieties to be delivered to a human body by the carrier 22, such as antibodies, peptides, DNAs, RNAs, oligonucleotides, or the like.

The moiety 26, in other examples, can be molecules and chelating agents with radioactive isotopes, ferromagnetic, and/or magnetic elements. In these examples, the compound 20 can be used as a contrast agent for medical imaging such as PET, SPECT, CT, MRI, etc.

In another example, the moiety 26 can include combinations of any of the example moieties 26 discussed above. In this example, the compound 20 can be used as a heterogeneous probe for biomedical detection and sensing.

Some nanomaterial carriers 22, and in particular, boron nitride (BN)-based nanomaterials, are known to be chemically inert. Therefore, it has been difficult to functionalize prior art nanomaterial carriers for covalent interactions with other structures. However, it has been discovered that carriers 22 such as the BN dot carrier shown in FIGS. 1A-C that have been subject to mechanical processing in solution or solvent, such as agitation, exhibit increased propensity to covalently interact with functional groups, such as functional group R on linker 24. The solution/solvent can be the same solution/solvent in which source material is treated to form nanodots as discussed in more detail below, or a different solution/solvent. Furthermore, it has been discovered that mechanical processing of nanomaterial carriers improves the solubility of the nanomaterial carriers in aqueous solutions, which can improve biocompatibility. Additionally, mechanical processing cuts carrier material into smaller pieces which can be desirable when forming dots, for example. Agitation can be accomplished by homogenizer and/or sonication, such as tip sonication or bath sonication, for instance.

Referring now to FIG. 1B, mechanical processing results in carrier 22 with imperfections 23. During mechanical processing in solution/solvent, imperfections 23 form on the carrier 22 such that localized polarities or charges are formed at the imperfections 23. Polar or charged groups from the solution/solvent interact with the localized polarities or charges at the imperfections. For the example, in the example of FIG. 1B, the carrier is an h-BN nanodot carrier 22. In this particular example, imperfections 23 are disruptions in the hexagonal structure of the boron nitride material, which disruptions have localized polarity imbalances. For example, for certain solvents/solutions, hydroxyl groups from the solvent/solution may interact with the imperfections 23, though other solvents/solutions may have other polar or charged groups that can interact with the localized imperfections 23, such as amino, carboxylic acids, or aldehyde groups, depending on the processing and type of solvent/solution.

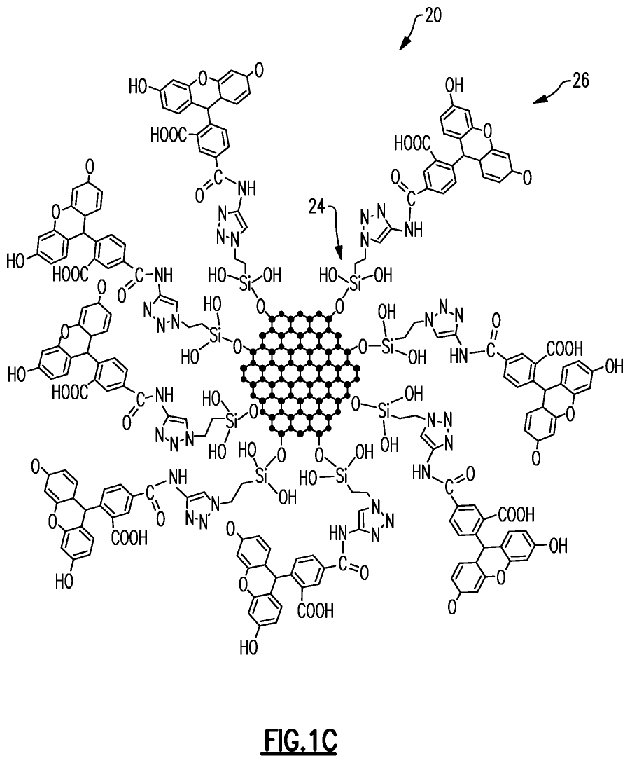

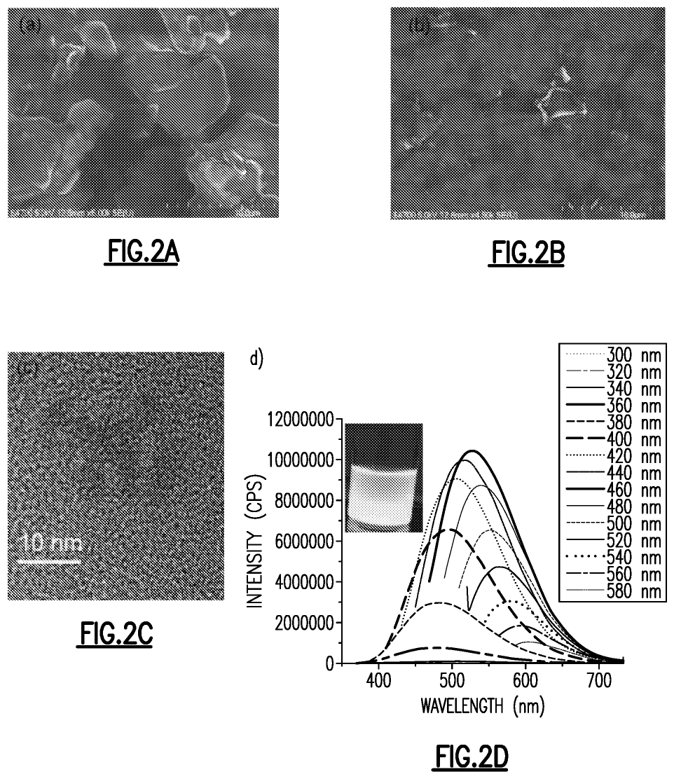

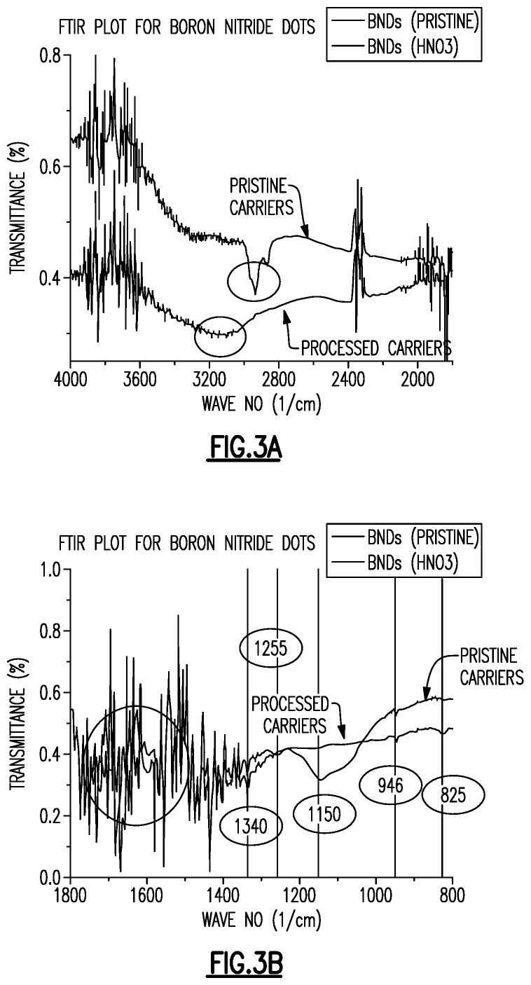

In one particular example method of making carriers 22, h-BN powder is treated in dimethylformamide (DMF) or another polar solution/solvent for two to four hours by using a homogenizer. In one example, the treatment in polar solvent is solvothermal (e.g., the solvent/solution is heated). In one example, the h-BN powder has an average particle size of between about 10-20 μm. In a particular example, the average particle size (e.g., diameter) is about 13 μm FIG. 2A shows images of example h-BN particles with average size of about 13 μm prior to treatment in DMF. The homogenizer causes the BN dot carriers 22 to become smaller and remain suspended in the DMF solution. In this example, after treatment in DMF solution, the BN dot carriers 22 become smaller, and the size is reduced to less than about 2-5 μm, as shown in FIG. 2B.

After the DMF treatment, the BN dot carrier 22 suspension undergoes an agitation treatment, such as sonication. In a particular example, the suspension is treated by bath sonication for 20-30 hours. The size of the BN dot carrier 22 is reduced to about 1-3 μm after sonication.

After the agitation treatment, the DMF/BN dot carrier 22 suspension is heat treated. In a particular example, the suspension is heated at 150° C. for 7 to 12 hours while stirring with a magnetic stir bar. The stir bar ensures that the BN dot carriers 22 remain suspended in the DMF solution.

Agitation and heat treatment result in carriers 22 with imperfections 23, as in the example of FIG. 1B.

After the heat treatment, the carriers 22 suspension is centrifuged to precipitate large particles. In a particular example, the suspension is centrifuged at 10,000 rpm for 10 minutes. In this example, the size of the carriers 22 in the suspension is about 2-10 nm after heat treatment and centrifugation, as confirmed by TEM (transmission electron microscopy) imaging shown in FIG. 2C. Furthermore, the carriers 22 are nearly invisible using SEM imaging, confirming that the carriers 22 are very small and have dimensions in the nano-scale. Generally, the carriers 22 have less than about 30 layers of h-BN, which corresponds to a thickness dimension of less than about 100 nm. The length/width dimensions are also less than about 100 nm. In a particular example, the carriers 22 have between about 4-8 layers of h-BN and have dimensions of about 2-10 nm.

After the centrifugation, the carriers 22 suspension undergoes solvent exchange. That is, the solvent (DMF) is switched for another solvent, water. Carriers 22 suspended in water are ready for biological applications or linking with moieties 26 to be carried, as discussed herein. Solvent exchange is accomplished as follows. DMF is evaporated into air by heating the suspension. In a particular example, the suspension is heated to 150° C. until the DMF is evaporated. After heating, the remaining carriers 22 are placed into a water/ethanol mixture. In a particular example, the water/ethanol mixture is 50% water and 50% ethanol. The carriers 22/water/ethanol mixture is then heated to evaporate the ethanol at an appropriate temperature as would be known in the art. In a particular example, DMF can be removed by vacuum treatment and then the carriers 22 can be suspended in water.

It has been discovered that making carriers 22 according to the above-described method leads to a production yield orders of magnitude higher than prior art methods. For example, for the method performed with 20-30 minutes of bath sonication, heat treatment for 7 to 12 hours while stirring with a magnetic stir bar, and centrifugation at 10,000 rpm for 10 minutes, the production yield is about 47%, as compared to the reported 1-26% for prior methods. Production yield is the weight percentage of h-BN bulk powder that become carriers 22 after the evaporation step discussed above.

For the example DMF solution, hydrocarbon groups or fragments from the solution interact with the localized polarities at the imperfections 23 of carriers 22, though other solutions may have other polar groups that can interact with the localized polarities, such as amino, carboxylic acids, aldehyde, etc. The carriers 22 can then undergo acid treatment according to any known method, which replaces the hydrocarbon groups or fragments with hydroxyl groups (—OH groups) at the imperfections 23 of carrier 22, which result in processed carriers (discussed in more detail below). Acid treatment also removes other contamination from the carriers 22, such as the hydrocarbon fragments of DMF. The processed carriers can then be linked to linkers 24 by any known chemistry that causes the R group of linker 24 to link covalently with the hydroxyl groups, to form functionalized carriers 220.

Carriers 22 made according to the above method are autofluorescent. That is, the carriers 22 have a measurable intrinsic fluorescence. FIG. 2D shows fluorescence intensity of the carriers 22 shown in FIGS. 2A-C formed by the above method. Without being bound by any particular theory, the autofluorescence may be related to imperfections 23 formed on the surfaces and edges of the carriers 22 during the above method. The imperfections 23 may bond with hydrocarbon fragments of DMF, including carbon-substituted N vacancy point defects, carbene structure at zigzag edges and BO₂⁻ and BO⁻ species. These imperfections 23 are expected to create a series of energy states near the edges of the valence and conduction bands of h-BN material.

FIGS. 1B-1C show synthesis of a compound 20. In this example, the carrier is an h-BN carrier 22 made by treating h-BN powder in a polar organic solvent which facilitates arrangement of the h-BN into a nanodot. Example polar organic solvents are dimethylformamide (DMF), N-Methyl-2-pyrrolidone (NMP), and ethanol. In a particular example, the carriers 22 are made according to the method described above.

In the example of FIG. 1B, h-BN dot carriers 22 made according to the method described above are treated with acid, here, nitric acid (HNO₃) to provide processed carriers 210. Acid treatment causes the attachment of —OH (hydroxyl) groups to the imperfections 23, which, as discussed above, have imbalanced polarities that are attracted to the —OH groups. FIGS. 3A-B show FTIR (Fourier Transform Infrared Spectroscopy) spectra for processed carriers 210 and non-functionalized (“pristine”) h-BN dot carriers 22. As shown in FIG. 3A, C—H stretching from DMF fragments at 2950 cm-1 of the pristine h-BN dot carriers 22 disappeared after the nitric acid treatment. A broad IR (infrared) band at 3100 cm⁻¹ is detected from the treated sample, which indicates that hydroxyl groups were introduced after acid treatment. There is a red shift on the —OH band due to the slight energy band change of zigzag edges of processed carriers 210 after DMF and contaminations were removed. The removal of these DMF fragments is also supported by the disappearance B—O (˜1255 cm-1), B—C or C—N (˜1150 cm-1) bonds shown in FIG. 3B. In other words, this FTIR analysis confirms the presence of hydroxyl groups on the processed carriers 210 by the presence of the expected peaks in the spectra.

The —OH groups attached to the imperfections 23 are themselves polar/charged. Turning again to FIG. 1B, the polar or charged groups (e.g., —OH groups, in this example) facilitate covalent interactions between the processed carrier 210 and the functional group R on linker 24. The polar groups also increase the hydrophilicity of the processed carrier 210 by facilitating polar or ionic interactions with water molecules or ions in the water. Therefore, the functionalized carrier 220 exhibits improved solubility dispersion in aqueous solution as compared to other carriers that do not include the processed carriers 210.

The processed carriers 210 have increased capacity for attaching to linkers 24 and thus moieties 26 due to the polar or charged groups as compared to non-functionalized carriers. More specifically, the polar or charged groups act as reactive sites for covalently linking the processed carrier 210 to linker 24 via functional group R. Accordingly, the brightness of the fluorophore 20 having a functionalized carrier 220 and a fluorescent entity 26 is higher than prior art fluorophores because the functionalized carrier 220 can be linked to multiple fluorescent entities 26. More generally, the functionalized carriers 220 can be linked to more moieties 26 than non-processed carriers.

In a particular example, the BN dot carriers 22 that are processed to form processed carriers 210 as discussed above have 4 layers of h-BN that are each about 2.5 nm in diameter. Each layer can bond to 10 or more linkers 24 and fluorescent entities 26 or other moieties 26 after processing as discussed above. Thus, the example processed carriers 210 can bond to 40 or more linkers 24 and fluorescent entities 26 to form a fluorophore. The fluorophore 20 is thus 40 or more times brighter than a carrier with a single fluorescent entity. For branched linkers (n branches), the intensity will be as larger as 40n times that of a carrier with a single fluorescent entity.

Turning again to FIG. 1B, an example triethoxysilane linker 24, which in this particular example is 3-(Azidopropyl)triethoxysilane, is linked to the processed carrier 210. In this example, the R group is a ethoxy silane group and the R′ group is an azide group. As shown in FIG. 1B, the R group is reactive with processed carrier 210 at imperfections 23 (and in particular, the polar-charged groups at imperfections 23) and the R′ group is reactive with moiety 26.

In other examples, the linker 24 is an amino-silane linkers. Other linkers 24 might have a variety of functional groups such as amino, carboxylic acid, succinimdyl ester, maleimide, carboimide, pyridyldithiol, haloacetyl, aryl azide, azide, alkyne, hydrazide and monosulfone groups. Those groups could be used for the conjugation of carriers 22 to dye, drug, or any targeting material. Cross-linkers which contain dual functional group can also be used to obtain functional group to conjugate linkers 24 to other entities such as dye, peptide, oligonucleotide, DNA, RNA, antibody, proteins, drugs or other nanoparticles. Those cross-linkers might be SMCC (succinimidyl 4-(N-maleimidomethyl)cyclohexane-1-carboxylate), sulfo-SMCC ((sulfo-succinimidyl 4-(N-maleimidomethyl)cyclohexane-1-carboxylate), AMAS (N-α-maleimidoacet-oxysuccinimide ester), BMPS (N-β-maleimidopropyl-oxysuccinimide ester), GMBS (N-γ-maleimidobutyryl-oxysuccinimide ester), sulfo-GMBS, MBS (m-maleimidobenzoyl-N-hydroxysuccinimide ester), sulfo-MBS, EMCS (N-ε-malemidocaproyl-oxysuccinimide ester), sulfo-EMCS, SMPB (succinimidyl 4-(p-maleimidophenyl)butyrate), sulfo-SMPB, SMPH (Succinimidyl 6-((beta-maleimidopropionamido)hexanoate), LC-SMCC succinimidyl 4-(N-maleimidomethyl)cyclohexane-1-carboxy-(6-amidocaproate), sulfo-KMUS (N-κ-maleimidoundecanoyl-oxysulfosuccinimide ester), SM(PEG)n where n=2,4,6,8,12,24 (PEGylated SMCC cross-linker), SPDP (succinimidyl 3-(2-pyridyldithio)propionate), LC-SPDP, sulfo-LC-SPDP, SMPT (4-succinimidyloxycarbonyl- alpha-methyl-α(2-pyridyldithio)toluene), PEGn-SPDP (where n=2,4,12, 24), SIA (succinimidyl iodoacetate), SBAP (succinimidyl 3-(bromoacetamido)propionate), SIAP (succinimidyl (4-iodoacetyl)aminobenzoate), sulfo-SIAP, ANB-NOS (N-5-azido-2-nitrobenzoyloxysuccinimide),sulfo-SANPAH (sulfosuccinimidyl 6-(4′-azido-2′-nitrophenylamino)hexanoate), SDA (succinimidyl 4,4′-azipentanoate), sulfo-SDA, LC-SDA, sulfo-LC-SDA, SDAD (succinimidyl 2-((4,4′-azipentanamido)ethyl)-1,3′-dithiopropionate), Sulfo-SDAD, DCC (N,N′-Dicyclohexylcarbodiimide), EMCH (N-ε-maleimidocaproic acid hydrazide), MPBH (4-(4-N-maleimidophenyl)butyric acid hydrazide), KMUH (N-κ-maleimidoundecanoic acid hydrazide), PDPH (3-(2-pyridyldithio)propionyl hydrazide), PMPI (p-maleimidophenyl isocyanate), SPB (succinimidyl-[4-(psoralen-8-yloxy)]-butyrate), or other known linkers.

In the example of FIG. 1B, the moiety 26 is a fluorescent entity, and in particular, is FITC (fluorescein isothiocyanate), which is a green dye. FITC can be conjugated to the linker 24 at R′ by any known chemistry. For instance, for the azide-silane linker 24 of FIG. 1B, a copper(I)-induced click reaction can be performed to covalently bond the R′ group of the linker 24 to an alkyne group of FITC.

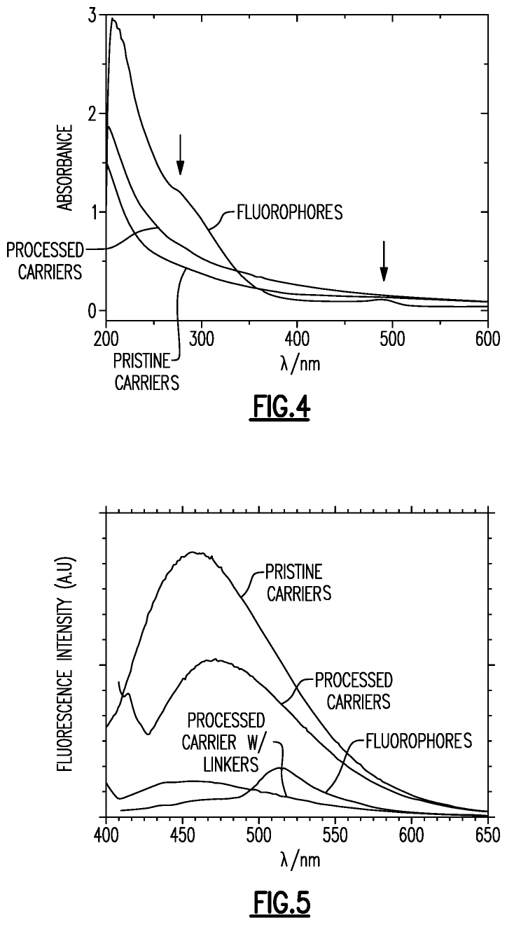

FIG. 4 shows the absorbance spectra of the example fluorophore 20 of FIG. 1B. The characteristic absorbance signal of FITC at around 490 nm and the peak at 280 nm attributed to aromatic triazole (shown at the arrows) is present in the fluorophores 20, confirming conjugation of the processed carrier 210 with the linker 24 and FITC entity 26. FIG. 4 also shows the absorbance spectra of pristine carriers 22 and processed carriers 210 for comparison.

FIG. 5 shows fluorescence intensity of the example fluorophore 20 of FIG. 1B after excitation with 492 nm irradiation. The fluorophore 20 emits at 515 nm, the characteristic emission signal of FITC molecules. This confirms that FITC molecules are covalently conjugated on the fluorophores 20. Fluorescence intensity of pristine carriers 22, processed carriers 210, and processed carriers 210 with linkers 24 (i.e., functionalized carrier 220) is also shown for comparison.

The same chemistry (e.g., copper (I)-induced click reaction discussed above) or other known chemistries can be applied to conjugate various fluorescent entities 26 that contain alkyne functional group such as sulforhodamine alkyne, sulfo-cy5.5 alkyne, etc. to the processed carrier 210 via linkers 24. Other moieties 26 such as alkyne-polyethylene glycol, alkyne antibodies, etc. can also be conjugated to the processed carrier 210 via linkers 24 using the same chemistry or other known chemistries. For example, alkyl antibodies can made by reducing an antibody using DTT (Dithiothreitol), which results in reduced sulfuhydryl groups, which can then be connected to with maleimide-PEG4-alkyne or another alkyne-containing moiety according to known procedure. Other small molecules such as sugars, nitroxides, biotin, drugs, etc. or macromolecules, peptides, DNA, RNA sequences, proteins such as SA (streptavidin and its derivatives) can also be covalently connected to the functionalized BN carrier 210/linker 24 according to known methods.

Though the preceding description of processed carrier 210 is made with respect to h-BN dots, carbon dots, and other nanodots of layered materials (TMDCs, etc. as discussed above) can be linked to linkers 24 by chemical means, such as by acid treatment, and then linked to moieties 26, as discussed above.

### Example Experimental Method

### 1. Synthesis of BN QDs

BN powder was firstly exfoliated to nanosheets through a solvent exfoliation method as reported previously. Typically, 51.3 mg of BN powder and 30 mL of DMF were homogenized for 3 hours under stirring. Then it was kept under sonication at least for 24 h and then heated with stir bar for 9 hour at 150° C. Afterwards, the resulting suspension was centrifuged for 10 min at 10000 rpm to separate the centrifuge and supernatant. The faint yellow supernatant was the BN dots (average size 2-10 nm) dispersion confirmed with TEM. DMF was removed by using high temperature the furnace under vacuum. The BN dots were stirred overnight in concentrated HNO₃. Afterwards, it the mixture was neutralized by sodium hydroxide solution. It was purified through dialysis (by using MWCO 1 KDa dialysis bag). Then the sample was collected by freeze-drying.

### 2. Covalent Functionalization of BN Dots with 3-(Azidopropyl)triethoxysilane)

Freeze-dried powder was dispersed in ethanol and toluene. Afterwards, 3-(Azidopropyl)triethoxysilane) (60 μl) was added in mixture. The mixture was heated to reflux and stirred under nitrogen overnight. The solvent was removed through rotation evaporation and the residue was dispersed in 70% ethanol (RE dialysis tubing 1 kDa). After dialysis, azide-silane functionalized BN dots were obtained. The sample was used directly without removing solvent.

### 3. Connection BN Dots with FITC

The functionalized BN dots was mixed with FITC alkyne (10 nM), sodium ascorbate (7.2 μM) and copper sulfate (7.2 μM). The reaction was processing under room temperature overnight. The solvent was removed through rotation evaporation and was dispersed in 70% ethanol for dialysis purification (RE dialysis tubing 1 kDa). The sample was stored 4° C. for analysis.

The preceding description is exemplary rather than limiting in nature. Variations and modifications to the disclosed examples may become apparent to those skilled in the art that do not necessarily depart from the essence of this invention. The scope of legal protection given to this invention can only be determined by studying the following claims.

What is claimed is:

1. A compound, comprising:
a nanodot carrier;
a moiety; and
a linker having first and second functional groups, wherein the first functional group is covalently linked to the nanodot carrier, and the second functional group is covalently linked to the moiety.

2. The compound of claim 1, wherein the nanodot carrier is an h-BN nanodot carrier.

3. The compound of claim 2, wherein the nanodot carrier has dimensions between about 2-10 nm.

4. The compound of claim 3, wherein the nanodot carrier comprises less than 30 layers of h-BN.

5. The compound of claim 4, wherein the nanodot carrier comprises between about 4 and 8 layers of h-BN.

6. The compound of claim 4, further comprising a plurality of linkers and a plurality of moieties, wherein each layer of the nanodot carrier is linked to 10 or more linkers of the plurality of linkers, and wherein each linker is linked to a moiety of the plurality of moieties.

7. The compound of claim 1, wherein the nanodot carrier has at least one polar group, and wherein the first functional group is covalently linked to the nanodot carrier at the at least one polar group.

8. The compound of claim 7, wherein the at least one polar group is a hydroxyl (—OH) group.

9. The compound of claim 1, wherein the moiety includes at least one of a fluorescient entity, a biological molecule, a chelating agent, and combinations thereof.

10. A method of making a nanodot carrier, comprising:
mechanically processing nanodots in polar liquid to create imperfections on the nanodots; and
treating the nanodots to provide polar groups at the imperfections.

11. The method of claim 10, further comprising covalently linking a linker to the nanodot, the linker having first and second functional groups, wherein the first functional group covalently links to the polar group.

12. The method of claim 11, further comprising covalently linking a moiety to the second functional group.

13. The method of claim 12, wherein the moiety is a fluorescent entity.

14. The method of claim 10, wherein the mechanically processing includes agitation.

15. The method of claim 14, wherein the agitation is accomplished by sonication or by homogenizer.

16. The method of claim 10, wherein the treating is an acid treatment, and wherein the polar groups are hydroxyl (—OH) groups.

17. The method of claim 10, wherein the polar liquid is dimethylformamide (DMF).

18. The method of claim 10, further comprising precipitating the nanodot carriers after the mechanical processing by centrifuging the nanodot carriers and polar liquid.

19. The method of claim 18, further comprising exchanging the polar liquid with water after the centrifuging.

20. The method of claim 19, wherein the treating is an acid treatment, and wherein the treating is performed after the exchanging.
